(19) United States
(12) Patent Application Publication
(10) Pub. No.: US 2004/0116351 A1
(43) Pub. Date: Jun. 17, 2004

(54) Method for enhancing the natural reward system for exercise

(75) Inventor: Brian D. Halevie-Goldman, Walnut Creek, CA (US)

Correspondence Address: PILLSBURY WINTHROP LLP, Suite 2800, 725 South Figueroa Street, Los Angeles, CA, 90017-5406, US

(73) Assignee: FAST BALANCE, INC., San Rafael, CA

(21) Appl. No.: 10/730,627

(22) Filed: Dec. 8, 2003

Related U.S. Application Data
(60) Provisional application No. 60/431,255, filed on Dec. 6, 2002.
(60) Provisional application No. 60/468,041, filed on May 5, 2003.

(51) Int. Cl. A61K 38/05; A61K 31/198

(52) U.S. Cl. 514/18; 514/567; 514/19

(57) ABSTRACT

Methods of enhancing and prolonging the natural reward system for exercise by administering one or more opiate destruction-inhibitors alone or in combination with one or more neurotransmitter precursors. When people exercise, they can experience a “runner's high” or a state of euphoria, which has be found to be based on natural opioids. By enhancing and prolonging the “runner's high,” incentive to exercise and to continue exercising will be increased. Further methods include the addition of any of a number of additives, such as those conventionally used for weight loss and appetite suppression.

### FIELD OF THE INVENTION

This invention relates to methods for enhancing and prolonging the natural motivation or reward system for exercise. More specifically, it relates to the enhancing and prolonging the natural reward system for exercise with enkephalinase inhibitors, such as hydrocinnamic acid, D-phenylalanine and D,L-phenylalanine (DLPA).

### BACKGROLND

Experts on nutrition and weight loss disagree on many things but agree that the major factor in predicting sustained weight loss in formerly obese individuals is exercise. Studies of post-surgical recovery and freedom from complications point to the critical importance of early mobilization and exercise. Recovery from most neuropsychiatric illnesses is augmented and sustained by a program of physical exercise. This is supported strongly in the medical literature, even when the degree of physical activity was prospectively considered to be an obfuscating variable. Patients who autonomously or by the recommendation of a health care practitioner or counselor, who engaged in regular physical activity, had the best outcomes. Post-treatment analysis revealed, in terms of relief of symptoms, subjective expressions of wellbeing and functional improvement in interpersonal satisfaction, by self-report and collateral histories and significant improvement in academic and occupational accomplishment.

When people exercise, they can experience a “runner's high” or a mild state of euphoria While the actual state that they feel can vary for each individual there is a common feeling associated with the term “runner's high.” It is a pleasant state that, for example, a runner might experience after a certain distance. Through studies with athletes it has been found that endorphin and enkephalin levels increase with exercise. Endorphins and enkephalins are neurotransmitters that are chemically similar to morphine, and have pain-relieving properties that naturally occur in the brain. It has been realized that the brain responds to morphine and that morphine receptors are in the brain. Knowing that human cells have receptors for this drug suggests that the body produces its own morphine like substances. Thus, it has been discovered that the runner's high is based on natural opioids. Pre-treatment of a runner with opioid antagonists, such as naloxone or naltrexone, abolishes the runner's high. Endorphins and enkephalins are broken down by peptidases. These peptidases may be inhibited safely without affecting other important metabolic activities in the body. Studies have confirmed the theoretical notion that enzymatic blockade of these peptidases increases the duration of the runner's high.

Good doctors spend a lot of time encouraging people to exercise, with limited success. Editorials in medical doctors chide physicians for not recommending regular physical exercise enough. Doctors themselves are not necessarily good role models for healthy active balanced life-styles, and their morbidity, suicide rate and mortality rate should not be above the general population of people they minister to. How many doctors regularly tell patients to exercise?? How many feel that they really should get more exercise themselves? The average life span of a physician, allowing for socio-economic status, is lower than that of his/her average patient.

Exercise has many benefits. Exercise is an excellent anti-depressant, good for general metabolism, osteoporosis, fitness, cardiovascular as well as neurovascular function, good for diabetes, everything except certain late stages of chronic fatigue syndrome/fibromyalgia (CFS/FM), chronic sleep disturbance or exercise induced asthma. There are products that give people energy so that they want to exercise or so that they have an increased inclination to exercise. There is a need for a product that enhances the natural reward of exercising so that people feel better during and after exercise and are more inclined to continue their exercise programs.

### SUMMARY OF THE DISCLOSURE

The present invention provides methods and compositions for enhancing the natural benefits of exercise with enkephalinase inhibitors, such as hydrocinnamic acid, D-phenylalanine or D,L-phenylalanine.

The present invention further provides methods and compositions for enhancing the natural benefits of exercise by administering one or more opiate destruction-inhibitors alone or in combination with one or more neurotransmitter precursors. The methods or compositions may further include various additives such as neurotransmitter agonists, blockers, antagonists, releasers or degradation inhibiters. Further additives can include any conventional weight loss compound, appetite suppressants, bulk water-soluble fiber water-absorbers, such as psilium husks, thyroid activating medications, thermogenic additives, or ephedra and/or guarana (caffeine).

Other features and advantages of the invention will become apparent from the following detailed description, which illustrates, by way of example, various features of embodiments of the present invention.

### DETAILED DESCRIPTION

L-Phenylalanine is an essential amino acid, which is also a precursor for the synthesis of the neurotransmitters dopamine and norepinephrine. These neurotransmitters, as measured by their metabolites, HVA, DOPAC, and MHPG, are significantly altered during periods of intense exercise. While not wishing to be bound by any theory, it is believed that L-tyrosine inhibits the further tyrosine synthesis (by phenylalanine hydroxylase, thereby encouraging the channeling of D-PA to phenylethylamine (PEA), a neurotransmitter that increases exploratory behavior, and energy). PEA can also increase concentration and mental awareness, excitement, motivation, along with decreased appetite, and a thermogenic action.

Certain substances analogous to L-phenylalanine, for example, hydrocinnamic acid, D-phenylalanine, and D,L-phenylalanine have been shown to inhibit the degradation of both enkephalins and endorphins. Enkephalinase inhibitors of the present invention may be combined with an enkephalin-releasing agent, such as the dopaminergic herb, Mucunia pruriens. It will be recognized that while this invention is directed to the use of a substance which inhibits the destruction of endogenous neuropeptidyl opiates, especially in combination with dopamine, serotonin and/or GABA precursors, it may be also be beneficial to add various neurotransmitter agonists, blockers, antagonists, releasers, or degradation inhibitors.

The use of these precursors may be supplemented at appropriate stages of treatment with dopaminergic releasers, blockers, agonists or antagonists, or agents affecting the reuptake or degradation of dopamine, norepinephrine or epinephrine. However, and while not wishing to be bound by any theory, it is believed that, the entire range of dopaminergic activity including synthesis, and release is regulated to some degree by certain opioid peptides (e.g. enkephalins and endorphins). Centrally administered opioid peptides (endorphins and enkephalins) produce elevations in levels of catecholamines in blood plasma in animals and humans. In fact, blockade of presynaptic dopaminergic receptors results in an enhancement of B-endorphin release, showing a reciprocal relationship.

An embodiment of this invention is the use of substances that inhibit the destruction of neuropeptidyl opiates, “opiate destruction-inhibitors.” Examples include of such substances include, but are not limited to, hydrocinnamic acid, D-form mono amino acids, thiolbenzyl amino acids, particularly thiolbenzyl-phenylalanine, di- and tripeptides of essential amino acids in D-form, enkephalin fragments, oligopeptides or polypeptides, which comprise the dipeptides D-Phe D-Leu or D-Phe D-Met. The substances may also include any analogues or derivatives of these amino acids and peptides. Some examples of such substances are hydrocinnamic acid as a single amino acid or as a dipeptide combined with tyrosine or L-leucine, thiobenzyl-phenylalanine as a single amino acid or as a dipeptide combined with tyrosine or L-leucine, D-PA as a single amino acid or as a dipeptide combined with tyrosine or L-leucine (D-PA structurally can be considered to be D-alpha-amino-hydrocinnamic acid), or DLPA as a racemic mixture of amino acids, or any combination thereof.

The opiate destruction-inhibitors of the present invention may be administered to a patient alone or in combination with one or more neurotransmitter precursors. Neurotransmitter precursors include, but are not limited to the dopamine precursors L-Phe, L-dopa and L-Tyr, the serotonin precursors 5-hydroxytryptophan and L-Trp, and the GABA precursors, L-Glutamine, L-glutamic acid and L-glutamate. The Neurotransmitter precursors may be added in a neurotransmitter synthesis promoting amount that is chosen so that the composition containing the one or more opiate destruction-inhibitors and the one or more neurotransmitter precursors is effective in increasing the natural reward system of exercise. As examples, neurotransmitter precursors may also include D-PA as precursor of PEA or D-tyrosine. The addition of vitamin B6 (pyridoxine) would aid in the synthesis of PEA, and essential methylating factors would create a tendency to form methylated phenylethylamines, such as DMPEA (Dimethoxy-phenylethylamine). Methylated indoleamines such as dimethyltryptomine (DMT) may also be formed. These methylating factors would include the amino-acid, L-methionine, trimethylglycine (TMG) and the methyl forms of the vitamins, folate (5-Methyl-terahydrofolate) and B 12 (methylcobalamin). Examples of methylating factors are also shown in U.S. patent application Ser. No. 09/781,822, which is herein incorporated by reference. The addition of quercentin or passion flower extract would decrease the activity of the mono-amine oxidase (MAO) enzyme that normally breaks down the naturally activating substance, phenylethylamine.

The neurotransmitter precursors may include one or more dopamine precursor, serotonin precursors, and/or GABA precursor. The composition may consist essentially of an enkephalinase inhibitor, a dopamine precursor, a serotonin precursor and a GABA precursor. The composition may also consist essentially of D-Phe, L-Phe, L-Tyr, L-Trp, and L-Gln.

The composition may further comprise any one of a number of cofactors. One such cofactor is tyrosine as ergotropic and/or stabilium. It may further comprise cofactors that are appetite suppressants, such as, but not limited to, Gymnema sylvestri, 5HTP, Rodiola Rosea, and/or any ingredients that increase Leptin, CCK-A and B, and/or neurotensin, and/or any ingredients that decrease neuropeptides &, Neurotensin YY and Gallanin. The composition may further comprise cofactors that increase thermogenesis and metabolic enhancement such as tyr-tyr bipeptide known as thyronine (from thyroid glandular extracts), Gugulu, Coleus Forskohlii, Rhodiola Rosea, 7-oxo-DHEA, or related adaptogens, or any combination thereof. The composition may further comprise cofactors that increase lipolysis. The composition may further comprise cofactors to decrease adrenal function where it is over-active, restore normal adrenal functioning where there is adrenal exhaustion.

The composition of the invention can be used in conjunction with Ephedra, and it can also be used as an effective and safe alternative to Ephedra. The composition can be as effective as Ephedra.

Ephedra is known to increase one's energy to begin an exercise session and to sustain that energy, but is dangerous (some fatalities have occurred in young otherwise healthy people using this over-the-counter (OTC) supplement), and manufacturers of various weight loss and energy-enhancing products are removing it from their products, as the legal and liability insurance costs have escalated, while various state and federal legislatures are considering banning it, removing it's OTC status and having it be available by prescription only.

The method of administering the substance may be a daily dosage. For example, while not wishing to be bound by any theory, it is believed that the following daily dosages may be used: the opiate destruction-inhibitors may be administered in a daily dosage of about 150-15,000 mg and the neurotransmitter precursor may be administered in a daily dosage of about 9-90,000 mg (if the neurotransmitter precursor is L-Tyrosine, about 100-5,000 mg (if the neurotransmitter precursor is L-Tryptophan), and about 100-10,000 mg (if the neurotransmitter precursor is L-Glutamine). Acetyl-tyrosine is a natural more fat-soluble form of tyrosine, which crosses the blood-brain-barrier readily, and would also be useful in this context at doses of about 10-500 mg.

The composition of the invention may also be combined with cofactors such as Ephedra or guarana, a potent natural source of caffeine, with or without cardio-protective agents and/or anti-arrhythmic agents. It also possible to use the composition of the invention in place of ephedra and/or sources of methyl-xanthines, such as caffeine (as from the Brazilian herb, guarana), theophylline (high in fermented teas) and theobromine (found in chocolate and cocoa).

The method of the present invention may be varied for different health profiles. For example, and without limitation, a questionnaire including questions regarding health profiles may be presented to a potential user of the composition. The answers from the questions will be analyzed to determine the body type of the potential user and any health conditions of that user, for example, hyper- or hypo-thyroid conditions, yeast conditions, FFA deficiencies, Adrenal high and low, and food allergies. The composition would then be varied according to the health profile of the potential user.

As an example, the composition of the present invention may contain one or more of the following categories of components. Some examples of components which fall under the above categories are also included below, with some example dosages (of course, any dosage which achieves the desired result is acceptable). The lists are not intended in any way to be exclusive.

Category 1:

This category includes enkephalinase and/or endorphinase inhibitors to activate the exercise reward principle.

Ingredients in this category can be used alone or in combination. Some example ingredients are as follows: (a) Hydrocinnamic acid 200 mg, as a single amino acid or as a dipeptide combined with tyrosine or L-leucine; (b) Thiobenzyl-phenylalanine as a single amino acid or as a dipeptide combined with tyrosine or L-leucine; (c) D-PA 100-200 mg as a single amino acid or as a dipeptide combined with tyrosine or L-leucine (D-PA, structurally can be considered to be D-alpha-amino-hydrocinnamic acid); and (d) DLPA 200 mg 400 mg as a racemic mixture of amino acids.

Category 2:

This category includes ingredients that promote intracellular creation and maintenance of methylated forms of phenylethylamine (PEA), for example, (a) precursors for the synthesis of neurotransmitters, (b) product/substrate blockers, (c) enzyme enhancing agents to promote the synthesis of PEA, (d) methylating factors (which are described in U.S. patent application Ser. No. 09/781,822, incorporated herein by reference) such as L-Methionine, 5-Methyl-THF (Methyl-Folate), Methylcobalmin-Vitamin B12, Betaine-TMG, and CoEnzyme Q10 to enhance the methylation of PEA; (d) natural substances to increase the lifespan of the PEA in the body.

Ingredients in this category can be used alone or in combination. Some example ingredients are as follows: (a) D-PA (preferred to DLPA) as precursor of PEA (200 mg-2 g) (see 1(c)); (b) Tyrosine 100 mg-200 mg (ratio of Hydrocinnamic acid, Thiobenzyl-phenylalanine or D-PA to Tyrosine is preferably about 1:1; if DLPA is used the ratio is preferably about 2:1); (c) Vitamin B6 200 mg (10,000% or 100×RDA) to activate the enzyme L-aromatic amino acid decarboxylase (L-AAA) to synthesize PEA from its precursor, phenylalanine; (d) essential Methylating factors such as dimethoxyphenylethylamine (DMPEA), formed from L-dopa or methoxyphenylethylamine derived from Tyrosine; (e) Quercetin 50 mg-100 mg (at this dose range, Quercetin has reversible MAO-B inhibiting activity, with minimal MAO-A inhibiting activity, giving this a good safety profile, and would prolong PEA activity beyond it's usual 10 minutes lifespan).

An alternate embodiment includes the herb, Passionflower, which also has activity on Cholecystokinin (CCK-A and CCK-B). Another alternate embodiment would involve the pharmaceutical MAO-B inhibitor, Deprenyl or selegeline, at doses of 5-10 mg daily. More than that could lead to the potential complications of MAO-A inhibition. Tricyclic antidepressants such as imipramine, desipramine, amitriptyline or nortriptyline may substitute for selegeline as a functional MAO-B inhibitor.

Category 3 (Cofactors):

This category includes ingredients to energize towards physical activity (preferably without being cardiotoxic).

Ingredients in this category can be used alone or in combination. Some example ingredients are as follows: Tyrosine 100-200 mg as ergotropic (also has dual purpose, see 2b and 6). Alternative embodiment would include Stabilium.

Category 4 (Cofactors):

This category includes ingredients to decrease appetite and reduce cravings.

Ingredients in this category can be used alone or in combination. Some example ingredients are as follows: Appetite suppressants (for example, Gymnema sylvestri, 5HTP, Rhodiola Rosea); ingredients that increase Leptin, CCK-A and B, neurotensin and decrease Neuropeptides Y, Neurotensin YY and Gallanin.

Category 5 (Cofactors):

This category includes ingredients that enhance thermogenesis.

Ingredients in this category can be used alone or in combination. Some example ingredients are as follows: Thermogenesis and metabolic enhancement (Candidates may include some tyr-tyr bipeptide, known as thyronine, available naturally from thyroid glandular extracts), Gugulu, Coleus Forskohlii, Rhodiola Rosea 50 mg, 7-oxo-DHEA) or related adaptogens.

Category 6 (Cofactors):

This category includes ingredients that improve concentration.

Ingredients in this category can be used alone or in combination. Some example ingredients are as follows: Tyrosine, see 2(b) and 3.

Category 7 (Cofactors):

This category includes ingredients that enhance lipolysis. Ingredients in this category can be used alone or in combination. Some example ingredients are as follows: CNOF-L-Leucine, Curcuminoids (see p. 117 of the “PDR for Nutritional supplements” (Sheldon Saul Hendler)), Butcher's broom, cardamon, cayenne, cinnamon, garcinia cambogia, ginger, mustard seeds (which also can improve digestion and aid fat metabolism), Banaba Leaf (Lagerstroemia speciosa. Glucosol, Regulin—Extract of leaf lowers blood sugar and reduce accumulation of triglycerides. It further can induce transport of glucose into cells and lower fat deposition in liver resulting from reduced triglyceride accumulation), Gamma Oryzanol (a substance extracted from rice bran oil that has a variety of metabolic effects, including increased endorphin release, antioxidant activity, lipotropic action, stress reduction, GH stimulation, increased growth, and improved recovery—Ferulic acid (FRAC) is part of the gamma-oryzanol molecule and is also available as a supplement), Policosanol 10 mg (which can lower cholesterol), Guggulu (Commiphora mukul—has anti-inflammatory and lipid-lowering actions; also can lower cholesterol with minimal side effects and is useful for weight loss, as this herb detoxifies fat cells); and Coleus forskohlii (which can stimulate Lipolipase A (Hormone-sensitive lipase)).

Category 8 (Cofactors):

This category includes ingredients to balance out adrenal functioning. Such ingredients can decrease adrenal function where it is over-active and restore normal adrenal functioning where there is adrenal exhaustion. The basic formulation can be added to product ingredients to create (A): a product for those with high adrenal (cortisol) activity, and (B): a product for those with low adrenal activity (low cortisone, Addisonian or adrenal exhaustion syndrome).

Ingredients in this category can be used alone or in combination. Some example ingredients are as follows: DHEA (or 7 oxo-DHEA) or pregnenolone, High dose Vitamin C (about 1 g-10 g), High dose Vitamin B5 (Pantothenate) (about 10 mg-100 mg) with or without Pantothine, DeGlycyrrhinated Licorice (DGL) licorice root Glycorhiza Glabra, Yerba Mate (has analeptic, diuretic, positively inotropic, positively chronotropic, glycogenolytic and lipolytic effects), Bladder wrack, borage seed (Borago officinalis), hawthorn berry, sarsaparilla (there's some evidence that sarsaparilla stimulates the adrenal glands and improve thyroid function), L-Glutamine (Extended release (ER)) form), CNOF-L-Leucine, Gymnema sylvestre (which diminishes the sensitivity of the taste buds for sweets, helps modulate the production of insulin, thereby contributing to the stabilization of energy, can stimulate the beta cells in the pancreas to produce insulin in insulin-dependant diabetics, and reduces blood sugar, glycosylated haemoglobin and glycosylated plasma proteins when used for 18-20 months. Gymnema Sylvestre leaf and leaf extract may be standardized to (leaf 25% gymnemic acids) 84.3 mcg (for hyperglycemia, but to be used very carefully with insulin or other hypoglycemics)), Yerba Mate, dandelion root (which can improve carbohydrate metabolism), Banaba in a dose of 10-50 mg per day, Chromium (trivalent) polynicotinate, GTF or glycinate or picolinate 200 mcg, Goat's rue (Galega officinalis)) tincture, and Devil's club (Oplopanax horridum) tincture.

The composition may also contain any other co-factors, such as tricyclics that inhibit MAO-B (which may potentiate the composition of the invention if it contains DLPA, Tyrosine and/or B6).

In one embodiment, the composition may include one or more components under category 1. It may further include one or more components under category 2. It may include, in addition to or instead of components under category 2, one or more components from one or more of the remaining categories (3-8).

### Experimental Data

A version of the composition of the invention, containing DLPA, tyrosine, Rhodiola rosea, eluthrococcus senticosis, the methylating factors (L-methionine, trimethylglycine (TMG) and the methyl forms of the vitamins, folate (5-Methyl-terahydrofolate) and B12 (methylcobalamin) and other B vitamins, was administered to 98 subjects. The formulation was administered in the following amounts in milligrams:

| | |
|---|---|
| DL-Phenylalanine | 200 |
| L-Tyrosine | 100 |
| Acetyl-L-Tyrosine | 20 |
| L-Glutamine | 200 |
| Mucuna Pruriens | 80 |
| Pregnenalone | 12 |
| Rhodiola Rosea | 200 |
| Chromium Picolinate | 0.2 |
| L-Methionine | 600 |
| 5-Methyl-THF (Methyl-Folate) | 0.10 |
| Methylcobalamin-Vit B12 | 0.10 |
| Betaine-TMG (Trimethyl- | 400 |
| Glycine) | |
| Pyridoxine (Vitamin B6) | 60 |
| CoEnzyme Q10 | 20 |

95 subjects described benefiting from the product. Among these were 16 patents with Chronic Fatigue Syndrome. Most reported feeling more alert and alive, especially after they exercised while on the product. Other reports included feelings of clearheadedness and well-being, more mental clarity, better physical and mental energy, improved focus, improved mental efficiency in handling difficult problems, feeling more ‘here and now’ and more able to be ‘present’ with a partner or spouse. A not uncommon report was describing feeling better than he/she felt for a long time. Two individuals used the term, ‘rejuvenated’.

120 people have been provided with samples of a composition of the invention. Eight people were seen for a one-time consultation, and no feedback is available at present, but it will be pursued. 14 people had not yet tried the composition, before exercising but have promised to do so. Feedback was obtained from 98 subjects so far. Three of these reported no significant effect. Two of these admitted trying the composition, but with only a minimal exercise effort. One subject (with Chronic Fatigue Syndrome and fibromyalgia) felt she had exercised sufficiently well, and felt her usual intense discomfort after exercising.

Below is the occurrence among the subjects of certain benefits relating to the natural rewards of exercising.

| | | |
|---|---|---|
| 1. | Decreased food cravings, | 75% |
| 2. | Increased mental energy | 80% |
| 3. | Increased physical energy | 66% |
| 4. | Feeling more ‘present’ | 50% |
| 5. | Enhanced desire to exercise | 60% |
| 6. | Enhanced mood after exercising | 85% |
| 7. | Enhanced enjoyment of exercise | 90% |
| 8. | Improved concentration and clarity | 80% |

1. Exercise Reward Composition vs. Ephedra

Of those subjects who have had experience with the use of Ephedra, without exception, they reported the superiority of the sample exercise reward composition over Ephedra with respect to the following parameters:

1. Appetite suppressant effect without causing stomach discomfort or nausea.

2. Physically energizing effect without causing irritability, agitation, heart palpitations, tachycardia or cardiac arrhythmia.

3. Thermogenic without destabilizing blood sugar. No rebound effects.

4. Mood enhancing without euphoria, mania or elation. The consensus was that it would bring about a pleasant uplifting mood, without causing the sort of euphoria that would lead to addiction.

Adverse Reactions:

One adverse reaction occurred when one subject gave some product to her father with Alzheimer's, who then proceeded to wander away much further than he ever had.

A rather sedentary overweight woman in her 40s, enrolled in a college, found after using the ExeReward capsules in the morning and exercising that morning, found it hard to sit down for her classes and then study, as she felt this urge to exercise again. This problem abated over the next couple of days, and she was able to find a sensible lifestyle balance between her studying, her family responsibilities and her prescribed exercise program.

Evaluations of Young Adults with Experience of using Ephedra Regularly:

The sample exercise reward composition was described as not as immediately alerting as Ephedra. It showed superior efficiency to ephedrine with respect to appetite suppression. There appeared to be less destabilization of blood sugar levels, such that there was less rebound food cravings and agitation. The sample exercise reward composition subjectively had less immediate energizing (ergotropic) effect, but, unlike Ephedra products, had no rebound effect into a state of food cravings and lowered metabolism (negatively thermogenic). The sample reward composition showed much less cardiological effects (not nearly as positively inotropic and chronotropic). Slightly less immediate effect on ADD symptoms, but a more natural feeling was reported and a more sustained action on concentration and freedom from distractibility.

Other Ephedra Subjects:

On Ephedra, a number of subjects reported feeling overly anxious, having a quicker temper and being a lot more impulsive. More than half agreed that Ephedra didn't do much for appetite. Some described feeling that they were thinking more clearly. A few mentioned sweating a lot. Two subjects reported feeling as if their heart wasn't beating correctly, and that their heartbeats were irregular.

One subject particularly had liked the Ephedra, at first but it seemed to become addicting, so he quit. On it he felt he “had an agenda, for specific tasks, it organized me”, although he was more spontaneous, more disciplined, more mental energy. He described less endurance without it. He started doing it when guy at the nutrition store said it would give him a good kick for the work-outs (that for him turned out to be very accurate). He took Ephedra and caffeine to ‘spark it’, in the morning. Once he felt like that he wanted to feel like that again, (therefore it was addicting). He described feeling snappy, lethargic and moody when it was coming down. He said, “If I didn't take it, the next day would be bad. I'd have to keep taking it or else I'd get moody”. A coke 3× a day gave him a similar effect. He stopped (but continued the caffeine) and was feeling weird, and to deal with it he went out to ‘numb it’ by drinking, and that's when he got into trouble.

Generally it seemed that family physicians tried to get their patients off the Ephedra, and suggested non-Ephedra-containing products, or prescribed alternatives, such as medications they considered safer prescription appetite suppressants (e.g. phentermine, phenmetrazine), stimulating anti-depressants (e.g. bupropion (Wellbutrin), or neurostimulants (e.g. methylphenidate (Ritalin)).

Post-Exercise (Exhilaration).

Placebo: In evaluating the sample exercise reward composition vs. placebo, on the enhancement effect on the runner's high, our studies were complicated by our finding that there was a stronger than expected placebo effect (on young fairly healthy people) effect with exercise producing positive effects on mood and energy without the sample exercise reward composition (40%). Most positive responders were somewhat older and were more overweight or were compromised in terms of their physical health in various ways. 80% of those in the placebo-controlled trial on the sample exercise reward composition reported improved mental clarity and energy, beyond that which they experienced with exercise without the sample exercise reward composition pre-treatment.

### Pharmacology

Absorption: amino-acid active transport. Crucial ingredients are well-absorbed. The product is best taken on an empty stomach, since the constituent amino-acids would be subject to competition from other amino-acids from the digestive breakdown of proteins in food. No problems are anticipated with respect to the pharmacokinetics, metabolism, and excretion. Interactions, Allergic reactions are a remote possibility. The chances of an allergic reaction to the herb Rhodiola rosea, which may produce urticaria (hives), is believed to be less than one in 300,000.

Phenylethylamine:

Continually synthesizing PEA is better than administering PEA, since PEA usually lasts no longer than 10 minutes in the body.

There are available reference about lack of cardiac stimulation of phenylethylamine. (Sabelli)

Ephedrine:

Unlike Ephedra which creates a ‘Short-term energize and then crash’ effect, the present invention relies on natural enzyme systems to rid the body of excess adrenergics.

Nonetheless, the present invention looks promising in preliminary trials as a therapeutic agent for increasing mental and physical energy, decreasing appetite and for treating Attention Deficit Disorder (ADD).

Amphetamine:

Ephedrine is a second rate amphetamine. The amphetamine effect a useful model for a desirable component of a weight loss product. Amphetamine has many disadvantages. It has a restricted legal status as a controlled substance, it is illegal in many countries, it has an addicting potential at high doses, it has cardiac stimulating and arrhythmogenic effects, it has limited duration of action, with potential rebound effects, and it has a potential to produce mania or a psychotic state clinically indistinguishable from paranoid schizophrenia. Ephedrine's legal status is currently in flux, whereas it shares the other disadvantages of amphetamines.

Some desirable properties of amphetamines include its anorectic effect, its locomotor enhancement and its action of increasing concentration and alertness.

Amphetamine is a synthetic phenylethylamine with a methyl group in alpha position rendering it resistant to the enzyme mono-amine oxidase (MAO).

Amphetamine-like substances are safe as long as one has access to their body's natural restraints. One of these constraints is the enzyme, MAO (mono-amine oxidase), which metabolizes biogenic amines such as serotonin, nor-epinephrine, epinephrine and dopamine into relatively inactive metabolites. If one loosens these constrains, as can be done with MAO Inhibitors, one has to be careful to avoid substances in OTC preparations, particularly sympathomimetic or adrenergic decongestants such as pseudo-ephedrine, foods that contain the amino-acid, tyramine, particularly cheese, wine and others, and many pharmaceuticals, especially anti-depressants and anti-Parkinsonian drugs.

Add:

An earlier version of the composition of the present invention began as a treatment for Attention Deficit Disorder (ADD). It was designed to increase mental focus and agility, while diminishing physical hyperactivity.

This is one anecdote of an earlier version of this product designed to improve focus only, given to an athlete, who wanted to have more focus on his workout. This therapeutic trial was a complete failure. In his words, “After I took those two capsules, I didn't feel like working out at all. In fact I felt like sitting down and reading a book. This hadn't happened to me in years!”

Essentially, one has to find the right ratio of ingredients and co-factors, to achieve a balance of NE (nor-epinephrine) to DA (dopamine) in the brain, that would create an energizing effect to the mind AND the body.

Dopamine and Norepinephrine Ratio:

DA-1 and NE Product. DA:NE::2:1.

The enzyme that converts DA to NE in the brain, Dopamine-Beta Hydoxylase (DBH) is a copper-dependent enzyme, also requiring the presence of certain co-factors, such as Vitamin C. A copper-cheilate of vitamin, with as little as 0.25 mg elemental copper can (at least in vitro) make a huge difference to DBH activity. Clinical studies suggest that this principle holds in vivo as well.

To determine DA:NE ratio.

Monitor NE Effect, DA Effect:

1. Plethysmography.

2. EEG alerting.(research)

Tyrosine is used predominantly as a break rather than an accelerator.

Hydrocinnamic Acid:

This is a naturally occurring metabolite of the catecholamines. L-Phenylalanine (L-PA) can be thought of structurally as L-alpha-amino-hydrocinnamic acid, whereas D-Phenylalanine (D-PA) can be thought of as D-alpha-amino-hydrocinnamic acid.

Hydrocinnamic acid has physico-chemical properties that enable it to be incorporated into the enkephalin or endorphin at the terminal position where it interferes with the attachment of the enkephalinase enzyme that breaks it down.

Hydrocinnamic acid appears to be superior to D-phenylalanine (D-PA) competes with L-phenylalanine (L-PA) for incorporation into the enkephalins and endorphins. A racemic mixture of L-PA and D-PA, known as DLPA would be acceptable, but D-PA alone would be preferable since it incorporates into the enkephalins and endorphins, rendering them resistant to immediate breakdown, and D-PA is a better precursor to PEA than the L-PA form. The most efficacious embodiment of an enkephalinase inhibitor would be a dipeptide consisting of the catecholamine metabolite hydrocinnamic acid and the amino acid tyrosine. Another dipeptide consisting of D-PA and tyrosine would be similarly effective. These dipeptides and other oligopeptides can be created from natural food supplements, such as amino acids, by simple cooking procedures.

Therefore, hydrocinnamic acid, and related alkaloids, derived from natural sources, can be incorporated into the terminal ends of enkephalins and endorphins, such that it is relatively immune from breakdown by the peptidase enzymes, and so when these feel-good chemicals are produced during exercise, this ‘runners' high’ is intensified and prolonged. If two capsules of this product is taken on an empty stomach about 12 hour before exercise, the runners' high, instead of lasting for 10 minutes, lasts for 10 hours.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

We claim:

1. A method of enhancing the natural reward system for exercise, the method comprising:
administering to a patient an opiate destruction-inhibitor.

2. The method of claim 1, wherein the opiate destruction-inhibitor is administered to the patient prior to exercise by the patient.

3. The method of claim 1, whereby the patient's energy is increased.

4. The method of claim 1, wherein the opiate destruction-inhibitor is selected from the group consisting of hydrocinnamic acid, a D-form mono amino acid, a thiolbenzyl amino acid, a dipeptide of essential amino acids in D-form, a tripeptide of essential amino acids in D-form, an enkephalin fragment, an oligopeptide, a polypeptide, and DLPA.

5. The method of claim 2, wherein the opiate destruction inhibitor is a dipeptide comprising a moiety selected from the group consisting of tyrosine and L-leucine.

6. The method of claim 2, wherein the thiolbenzyl amino acid is thiolbenzyl-phenylalanine.

7. The method of claim 2, wherein the D-form mono amino acid is D-PA.

8. The method of claim 2, wherein the oligopeptide and polypeptide comprise a dipeptide selected from the group consisting of D-Phe, D-Leu, and D-Phe D-Met.

9. The method of claim 1, further comprising administering to the patient a neurotransmitter precursor.

10. The method of claim 7, wherein the neurotransmitter precursor is selected from the group consisting of a dopamine precursor, a serotonin precursor, and a GABA precursor.

11. The method of claim 8, wherein the dopamine precursor is selected from the group consisting of L-Phe, L-dopa, and L-Tyr.

12. The method of claim 8, wherein the serotonin precursor is selected from the group consisting of 5-hydroxytryptophan and L-Trp.

13. The method of claim 8, wherein the GABA precursor is selected from the group consisting of L-Glutamine, L-glutamic acid, and L-glutamate.

14. The method of claim 1, further comprising administering to the patient a dopamine precursor, a serotonin precursor and a GABA precursor.

15. The method of claim 1, further comprising administering to the patient Ephedra.

16. The method of claim 1, further comprising administering one or more cofactors.

17. The method of claim 13, wherein the one or more cofactors is selected from the group consisting of N-acetyl-tyrosine, coleus forskohlii, L-glutamine, mucuna pruriens, rhodiola rosea, pregnenalone, chromium picolinate, chromium polynicotinate, L-Methionine, methylcobalamin-vitamin B12, betaine-TMG, 7-oxo-DHA, acetyl-1-carnitene, green tea catechins, and L-theanine.

18. The method of claim 13, wherein the cofactor enhances the natural production of an activating neurotransmitter.

19. The method of claim 15, wherein the neurotransmitter is phenylethylamine.

20. The method of claim 1, wherein the opiate destruction-inhibitor is administered daily in a daily dosage of about 150 to about 15,000 mg.

21. The method of claim 1, wherein the opiate destruction-inhibitor is administered daily and is selected from the group consisting of hydrocinnamic acid in a daily dosage of about 200 mg, thiobenzyl-phenylalanine in a daily dosage of about 50 mg-100 mg, D-PA in a daily dosage of about 100 to about 200 mg, and DLPA as a racemic mixture of amino acids in a daily dosage of about 200 to about 400 mg.

22. The method of claim 7, wherein the neurotransmitter precursor is administered daily in a daily dosage of about 25 mg to about 10,000 mg.

23. The method of claim 7, wherein the neurotransmitter precursor is administered daily and is selected from the group consisting of L-Tyrosine in a daily dosage of about 9 to about 90,000 mg, L-Tryptophan in a daily dosage of about 100 to 5,000 mg, L-Glutamine in a daily dosage of about 100 to about 10,000 mg, and acetyltyrosine in a daily dosage of about 10 to about 500 mg.

24. A method of enhancing the natural reward system for exercise, the method comprising:
administering to a patient D-Phe, L-Phe, L-Tyr, L-Trp and L-Gln.

25. A composition for enhancing the natural reward system for exercise comprising an opiate destruction-inhibitor and a precursor, wherein the precursor enhances the natural production of an activating neurotransmitter, in an amount pharmaceutically effective to enhance the natural reward system of exercise.

26. The composition of claim 25, wherein the composition is at least as effective as Ephedra in increasing energy in a patient.

27. The composition of claim 22, wherein the opiate destruction-inhibitor is selected from the group consisting of hydrocinnamic acid, a D-form mono amino acid, a thiolbenzyl amino acid, a dipeptide of essential amino acids in D-form, a tripeptide of essential amino acids in D-form, an enkephalin fragment, an oligopeptide, a polypeptide, and DLPA.

28. The composition of claim 22, wherein the opiate destruction inhibitor is a dipeptide comprising a moiety selected from the group consisting of tyrosine and L-leucine.

29. The composition of claim 22, wherein the thiolbenzyl amino acid is thiolbenzyl-phenylalanine.

30. The composition of claim 22, wherein the D-form mono amino acid is D-PA.

31. The composition of claim 22, wherein the oligopeptide and polypeptide comprise a dipeptide selected from the group consisting of D-Phe, D-Leu, and D-Phe D-Met.

32. The composition of claim 22, wherein the neurotransmitter precursor is selected from the group consisting of a dopamine precursor, a serotonin precursor, and a GABA precursor.

33. The composition of claim 28, wherein the dopamine precursor is selected from the group consisting of L-Phe, L-dopa, and L-Tyr.

34. The composition of claim 28, wherein the serotonin precursor is selected from the group consisting of 5-hydroxytryptophan and L-Trp.

35. The composition of claim 28, wherein the GABA precursor is selected from the group consisting of L-Glutamine, L-glutamic acid, and L-glutamate.

36. The composition of claim 21, further comprising a dopamine precursor, a serotonin precursor and a GABA precursor.

37. The composition of claim 25, further comprising Ephedra.

38. The composition of claim 22, further comprising one or more cofactors.

39. The composition of claim 33, wherein the one or more cofactors is selected from the group consisting of N-acetyl-tyrosine, coleus forskohlii, L-glutamine, mucuna pruriens, rhodiola rosea, pregnenalone, chromium picolinate, chromium polynicotinate, L-Methionine, methylcobalamin-vitamin B12, betaine-TMG, 7-oxo-DHA, acetyl-1-camitene, green tea catechins, and L-theanine.

40. The composition of claim 33, wherein the cofactor enhances the natural production of an activating neurotransmitter.

41. The composition of claim 35, wherein the neurotransmitter is phenylethylamine.

42. The composition of claim 22, wherein the composition comprises about 150 to about 15,000 mg of the opiate destruction-inhibitor.

43. The composition of claim 22, wherein the opiate destruction-inhibitor is selected from the group consisting of hydrocinnamic acid in an amount of about 200 mg, thiobenzyl-phenylalanine in an amount of about 25 mg-100 mg, D-PA in an amount of about 100 to about 200 mg, and DLPA as a racemic mixture of amino acids in an amount of about 200 to about 400 mg.

44. The composition of claim 22, wherein the neurotransmitter precursor is selected from the group consisting of L-Tyrosine in an amount of about 9 to about 90,000 mg, L-Tryptophan in an amount of about 100 to 5,000 mg, L-Glutamine in an amount of about 100 to about 10,000 mg, and acetyltyrosine in an amount of about 10 to about 500 mg.

45. A composition for enhancing the natural reward system for exercise consisting essentially of D-Phe, L-Phe, L-Tyr, L-Trp and L-Gln.
